(19) United States
(12) Patent Application Publication
(10) Pub. No.: US 2026/0018289 A1
(43) Pub. Date: Jan. 15, 2026

(54) METHOD AND DEVICE FOR PROVIDING DIAGNOSTIC INFORMATION ABOUT SPINAL DISEASES USING A NATURAL LANGUAGE PROCESSING MODEL RELATED TO SPINAL DISEASES

(71) Applicant: AllthatSpine Co., Ltd., Pohang-si (KR)

(72) Inventor: Gun CHOI, Pohang-si (KR)

(73) Assignee: AllthatSpine Co., Ltd., Pohang-si (KR)

(21) Appl. No.: 19/262,302

(22) Filed: Jul. 8, 2025

(30) Foreign Application Priority Data
Jul. 10, 2024 (KR) 10-2024-0090994

(51) Int. Cl. G16H 50/20; G06F 40/284

(52) U.S. Cl. CPC G16H 50/20; G06F 40/284

(57) ABSTRACT

Provided are a method and device for providing diagnostic information about spinal diseases using a natural language processing model related to spinal diseases. The method according to an embodiment includes receiving symptom information of a patient with spinal disease from the user terminal, wherein the symptom information of the patient with spinal disease includes a natural language sentence input by the user terminal; determining standardized information about the symptoms of the patient with spinal disease through a natural language processing model related to spinal diseases, based on the symptom information of the patient with spinal disease, wherein the spinal disease-related natural language processing model is a model trained to standardize symptom information of a patient with spinal disease based on chief complaints of a plurality of patients with spinal disease, wherein the standardized information about the symptoms of the patient with spinal disease includes standardized information on a body part where the symptom occurs, standardized information on a symptom type, and standardized information on symptom severity; determining diagnostic information about the patient with spinal disease based on the standardized information about the symptoms of the patient with spinal disease; and transmitting the diagnostic information about the patient with spinal disease to the user terminal.

### CROSS-REFERENCE TO RELATED APPLICATION

This application claims priority to Korean Patent Application No. 10-2024-0090994, filed on Jul. 10, 2024 in the Korean Intellectual Property Office, the disclosure of which is incorporated herein by reference.

### BACKGROUND OF THE DISCLOSURE

### Field of the Disclosure

Embodiments of the present disclosure relate to a technology for providing diagnostic information about spinal diseases, and more particularly, to a method and device for providing diagnostic information about spinal diseases using a natural language processing model related to spinal diseases.

### Description of the Related Art

Spinal diseases can significantly impact a patient's quality of life, causing pain, mobility issues, and various other health problems. To effectively diagnose and treat spinal diseases, it is essential to understand the patient's chief complaints. These chief complaints represent the primary reasons why the patient seeks medical attention and play a critical role in evaluating and diagnosing spinal conditions. However, there may be various difficulties in collecting and processing such chief complaints from patients with spinal diseases.

Chief complaints are typically recorded in unstructured sentence formats within electronic medical records (EMRs), and such unstructured data formats require substantial time and effort for collection and preprocessing. Consequently, this unstructured nature may hinder standardization and efficient analysis, leading to delays in building comprehensive datasets for machine learning purposes. Additionally, since different medical institutions may use varying terms and formats to describe similar symptoms, data processing tasks may become complex. Furthermore, even within the same institution, different medical professionals may use different terms to describe the same condition, resulting in reduced data consistency.

Meanwhile, the open information environment accessible via the internet can spread inaccurate medical knowledge, potentially having a negative impact on patient treatment. Therefore, a robust system is needed that can filter and deliver verified medical knowledge from experts to provide reliable diagnostic and treatment recommendations.

Accordingly, there is a need for a method and device for providing diagnostic information about spinal diseases using a natural language processing model related to spinal diseases.

### SUMMARY OF THE DISCLOSURE

Therefore, the present disclosure has been made in view of the above problems, and it is an object of the present disclosure to provide a method and device for providing diagnostic information about spinal diseases using a natural language processing model related to spinal diseases.

The technical problems to be solved by the embodiments are not limited to those described above, and other technical problems not mentioned may also be considered by those skilled in the art based on various embodiments to be described hereinafter.

In accordance with an aspect of the present disclosure, the above and other objects can be accomplished by the provision of a method of providing diagnostic information about spinal diseases to a user terminal by a server, the method including: receiving symptom information of a patient with spinal disease from the user terminal, wherein the symptom information of the patient with spinal disease includes a natural language sentence input by the user terminal; determining standardized information about the symptoms of the patient with spinal disease through a natural language processing model related to spinal diseases, based on the symptom information of the patient with spinal disease, wherein the spinal disease-related natural language processing model is a model trained to standardize symptom information of a patient with spinal disease based on chief complaints of a plurality of patients with spinal disease, wherein the standardized information about the symptoms of the patient with spinal disease includes standardized information on a body part where the symptom occurs, standardized information on a symptom type, and standardized information on symptom severity; determining diagnostic information about the patient with spinal disease based on the standardized information about the symptoms of the patient with spinal disease; and transmitting the diagnostic information about the patient with spinal disease to the user terminal.

### BRIEF DESCRIPTION OF THE DRAWINGS

The accompanying drawings, which are included as part of the detailed description to facilitate understanding of the embodiments, illustrate various embodiments and describe technical features of the embodiments in conjunction with the detailed description:

FIG. 1 is a diagram illustrating the configuration of an electronic device according to an embodiment;

FIG. 2 is a diagram illustrating the configuration of a program according to an embodiment;

FIG. 3 illustrates a system for assisting in the diagnosis of a patient with spinal disease using artificial intelligence according to an embodiment;

FIG. 4 illustrates a method in which a server provides diagnostic information about spinal diseases to a user terminal according to an embodiment;

FIG. 5 illustrates a spinal disease-related natural language processing model according to an embodiment; and

FIG. 6 illustrates a block diagram illustrating the configuration of a server according to one embodiment.

### DETAILED DESCRIPTION OF THE DISCLOSURE

The following embodiments are proposed by combining constituent components and characteristics of the embodiments according to a predetermined format. The individual constituent components or characteristics should be considered optional factors on the condition that there is no additional remark. If required, the individual constituent components or characteristics may not be combined with other components or characteristics. Also, some constituent components and/or characteristics may be combined to implement various embodiments of the present disclosure. The order of operations to be disclosed in the various embodiments of the present disclosure may be changed. Some components or characteristics of any embodiment may also be included in other embodiments, or may be replaced with those of the other embodiments as necessary.

In the description of the drawings, procedures or steps that may obscure the essence of the various embodiments are not described, and procedures or steps that can be readily understood by those skilled in the art are also omitted.

Throughout the specification, when a certain part is described as “comprising” or “including” a certain component, it is to be understood that, unless expressly stated otherwise, the component does not exclude the presence of other components, but rather may further include additional components. In addition, the terms such as “ . . . part”, “ . . . device”, and “module” as used in the specification refer to elements that process at least one function or operation, and may be implemented in hardware, software, or a combination of both. Moreover, the expressions “a”, “an”, “one”, “the”, and similar terms used in the context of describing various embodiments (particularly in the context of the claims below) may be interpreted to encompass both singular and plural forms, unless clearly specified otherwise or explicitly contradicted by the context.

Hereinafter, embodiments according to various examples will be described in detail with reference to the accompanying drawings. The detailed description presented below, together with the drawings, is intended to illustrate exemplary embodiments of the present disclosure and is not intended to represent the only form of embodiment.

In addition, specific terms used in the various embodiments are provided to facilitate understanding of the embodiments, and the use of such specific terms may be replaced with other forms without departing from the technical spirit of the present disclosure.

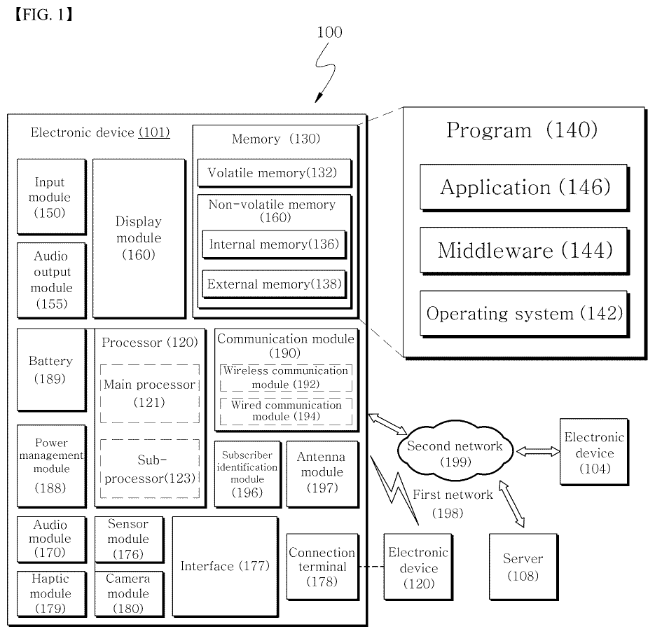

FIG. 1 is a diagram illustrating the configuration of an electronic device according to an embodiment.

FIG. 1 is a block diagram of the electronic device 101 in a network environment 100 according to various embodiments. Referring to FIG. 1, in the network environment 100, the electronic device 101 may communicate with an electronic device 102 via a first network 198 (e.g., a short-range wireless communication network), or may communicate with an electronic device 104 or a server 108 via a second network 199 (e.g., a long-range wireless communication network).

According to one embodiment, the electronic device 101 may communicate with the electronic device 104 through the server 108. According to one embodiment, the electronic device 101 may include a processor 120, a memory 130, an input module 150, an audio output module 155, a display module 160, an audio module 170, a sensor module 176, an interface 177, a connection terminal 178, a haptic module 179, a camera module 180, a power management module 188, a battery 189, a communication module 190, a subscriber identification module 196, or an antenna module 197. In some embodiments, at least one (e.g., the connection terminal 178) of the components of the electronic device 101 may be omitted, or one or more other components may be added. In some embodiments, some (e.g., the sensor module 176, the camera module 180, or the antenna module 197) of these components may be integrated into a single component (e.g., the display module 160). The electronic device 101 may also be referred to as a client, terminal, or peer.

The processor 120 may, for example, execute software (e.g., a program 140) to control at least one other component (e.g., hardware or software component) of the electronic device 101 connected to the processor 120, and may perform various data processing operations or computations. According to one embodiment, as at least a part of the data processing or computation, the processor 120 may store instructions or data, received from other components (e.g., the sensor module 176 or the communication module 190), in volatile memory 132, process the instructions or data stored in the volatile memory 132, and store the resulting data in non-volatile memory 134. According to one embodiment, the processor 120 may include a main processor 121 (e.g., a central processing unit or application processor) or a sub-processor 123 (e.g., a graphics processing unit, neural processing unit (NPU), image signal processor, sensor hub processor, or communication processor) that may operate independently or in conjunction with the main processor. For example, when the electronic device 101 includes both the main processor 121 and the sub-processor 123, the sub-processor 123 may consume less power than the main processor 121 or may be configured to specialize in a designated function. The sub-processor 123 may be implemented separately from or as part of the main processor 121.

The sub-processor 123 may, for example, control at least some of the functions or states related to at least one component (e.g., the display module 160, the sensor module 176, or the communication module 190) of the electronic device 101 in place of the main processor 121 while the main processor 121 is in an inactive state (e.g., sleep), or in conjunction with the main processor 121 while it is in an active state (e.g., executing an application). According to one embodiment, the sub-processor 123 (e.g., an image signal processor or a communication processor) may be implemented as part of another functionally related component (e.g., the camera module 180 or the communication module 190). According to one embodiment, the sub-processor 123 (e.g., a neural processing unit) may include a hardware architecture specialized for processing an artificial intelligence model.

An artificial intelligence model may be generated through machine learning. Such learning may be performed, for example, on the electronic device 101 itself, on which the artificial intelligence model operates, or via a separate server (e.g., the server 108). The learning algorithm may include, for example, supervised learning, unsupervised learning, semi-supervised learning, or reinforcement learning, but is not limited to these examples. The artificial intelligence model may include multiple artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), a deep Q-network, or a combination of two or more thereof, but is not limited thereto. The artificial intelligence model may include, in addition to or instead of a hardware architecture, a software structure.

The memory 130 may store various data used by at least one component (e.g., the processor 120 or the sensor module 176) of the electronic device 101. The data may include, for example, software (e.g., the program 140) and input data or output data related to instructions associated therewith. The memory 130 may include the volatile memory 132 or the non-volatile memory 134.

The program 140 may be stored in the memory 130 as software and may include, for example, an operating system 142, middleware 144, or an application 146.

The input module 150 may receive instructions or data, which may be used by a component (e.g., the processor 120) of the electronic device 101, from the outside of the electronic device 101 (e.g., from a user). The input module 150 may include, for example, a microphone, mouse, keyboard, key (e.g., button), or digital pen (e.g., stylus pen).

The audio output module 155 may output audio signals to the outside of the electronic device 101. The audio output module 155 may include, for example, a speaker or a receiver. The speaker may be used for general purposes such as multimedia playback or recorded audio playback. The receiver may be used for receiving incoming calls. According to one embodiment, the receiver may be implemented separately from or as part of the speaker.

The display module 160 may visually provide information to the outside of the electronic device 101 (e.g., to a user). The display module 160 may include, for example, a display, a hologram device, or a projector, and a control circuit for controlling such devices. According to one embodiment, the display module 160 may include a touch sensor configured to detect touch, or a pressure sensor configured to measure the intensity of force caused by the touch.

The audio module 170 may convert sound into an electrical signal or, conversely, convert an electrical signal into sound. According to one embodiment, the audio module 170 may acquire sound through the input module 150 or output sound through the audio output module 155, or through an external electronic device (e.g., the electronic device 102 (e.g., a speaker or headphones)) connected directly or wirelessly to the electronic device 101.

The sensor module 176 may detect the operating state (e.g., power or temperature) of the electronic device 101 or the environmental condition of the outside (e.g., user status), and generate an electrical signal or data value corresponding to the detected condition. According to one embodiment, the sensor module 176 may include, for example, a motion sensor, gyro sensor, barometric sensor, magnetic sensor, acceleration sensor, grip sensor, proximity sensor, color sensor, infrared sensor, biometric sensor, acoustic sensor, temperature sensor, humidity sensor, or illuminance sensor.

The interface 177 may support one or more designated protocols that allow the electronic device 101 to connect directly or wirelessly with an external electronic device (e.g., the electronic device 102). According to one embodiment, the interface 177 may include, for example, an HDMI (high definition multimedia interface), universal serial bus (USB) interface, SD card interface, or audio interface.

The connection terminal 178 may include a connector through which the electronic device 101 can be physically connected to an external electronic device (e.g., the electronic device 102). According to one embodiment, the connection terminal 178 may include, for example, an HDMI connector, USB connector, SD card connector, or audio connector (e.g., a headphone jack).

The haptic module 179 may convert electrical signals into mechanical stimuli (e.g., vibration or movement) or electrical stimuli that can be perceived by the user through tactile or kinesthetic sensation. According to one embodiment, the haptic module 179 may include, for example, a motor, a piezoelectric element, or an electrical stimulation device.

The camera module 180 may capture still images and video. According to one embodiment, the camera module 180 may include one or more lenses, image sensors, image signal processors, or flashes.

The power management module 188 may manage power supplied to the electronic device 101. According to one embodiment, the power management module 188 may be implemented as at least part of a power management integrated circuit (PMIC).

The battery 189 may supply power to at least one component of the electronic device 101. According to one embodiment, the battery 189 may include, for example, a non-rechargeable primary battery, a rechargeable secondary battery, or a fuel cell.

The communication module 190 may support the establishment of a direct (e.g., wired) communication channel or a wireless communication channel between the electronic device 101 and an external electronic device (e.g., the electronic device 102, the electronic device 104, or the server 108), as well as communication through the established channel. The communication module 190 may operate independently of the processor 120 (e.g., an application processor), and may include one or more communication processors that support direct (wired) or wireless communication. According to one embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication module). Among these communication modules, the applicable module may communicate with an external electronic device 104 through the first network 198 (e.g., a short-range communication network such as Bluetooth, wireless fidelity (Wi-Fi) direct, or IrDA) or the second network 199 (e.g., a long-range communication network such as a legacy cellular network, 5G network, next-generation communication network, the Internet, or a computer network such as a LAN or WAN). These various types of communication modules may be integrated into a single component (e.g., a single chip) or implemented as a plurality of separate components (e.g., multiple chips). The wireless communication module 192 may identify or authenticate the electronic device 101 within a communication network such as the first network 198 or the second network 199 using subscriber information (e.g., International Mobile Subscriber Identity (IMSI)) stored in the subscriber identification module 196.

The wireless communication module 192 may support post-4G technologies such as 5G networks and next-generation communication technologies, for example, new radio (NR) access technology. NR access technology may support high-speed transmission of large-volume data (eMBB: enhanced mobile broadband), minimized device power consumption and access by a large number of devices (mMTC: massive machine type communications), or high reliability and low latency (URLLC: ultra-reliable and low-latency communications). The wireless communication module 192 may support a high-frequency band (e.g., mmWave band) in order to achieve high data transmission rates. The wireless communication module 192 may also support various technologies for maintaining performance in high-frequency bands, such as beamforming, massive MIMO (multiple-input and multiple-output), full-dimensional MIMO (FD-MIMO), array antennas, analog beam-forming, or large-scale antennas. The wireless communication module 192 may support various requirements defined by the electronic device 101, an external electronic device (e.g., the electronic device 104), or a network system (e.g., the second network 199). According to one embodiment, the wireless communication module 192 may support a peak data rate (e.g., 20 Gbps or higher) for eMBB, a loss coverage (e.g., 164 dB or less) for mMTC, or a U-plane latency (e.g., 0.5 ms or less for each downlink (DL) and uplink (UL), or a round-trip of 1 ms or less) for URLLC.

The antenna module 197 may transmit signals or power to the outside (e.g., an external electronic device) or receive them from the outside. According to one embodiment, the antenna module 197 may include an antenna including a radiator formed of a conductor or conductive pattern provided on a substrate (e.g., a printed circuit board (PCB)). According to one embodiment, the antenna module 197 may include a plurality of antennas (e.g., an array antenna). In this case, at least one antenna suitable for a communication method used in a communication network such as the first network 198 or the second network 199 may be selected from among the plurality of antennas by, for example, the communication module 190. Signals or power may be transmitted or received between the communication module 190 and an external electronic device through the selected at least one antenna. In some embodiments, in addition to the radiator, another component (e.g., a radio frequency integrated circuit (RFIC)) may additionally be formed as part of the antenna module 197.

According to various embodiments, the antenna module 197 may form an mmWave antenna module. According to one embodiment, the mmWave antenna module may include a printed circuit board, a radio frequency integrated circuit (RFIC) disposed on or adjacent to a first surface (e.g., a bottom surface) of the printed circuit board and capable of supporting a designated high-frequency band (e.g., mmWave band), and a plurality of antennas (e.g., array antennas) disposed on or adjacent to a second surface of the printed circuit board (e.g., a top surface or side surface) and configured to transmit or receive signals in the designated high-frequency band.

At least some of the above components may be interconnected via communication interfaces between peripheral devices (e.g., a bus, general-purpose input and output (GPIO), serial peripheral interface (SPI), or mobile industry processor interface (MIPI)) and may exchange signals (e.g., instructions or data) with one another.

According to one embodiment, instructions or data may be transmitted or received between the electronic device 101 and the external electronic device 104 through the server 108 connected to the second network 199. Each of the external electronic devices 102 or 104 may be of the same or a different type compared to the electronic device 101. According to one embodiment, all or part of the operations executed by the electronic device 101 may be executed by one or more of the external electronic devices 102, 104, and 108. For example, when the electronic device 101 needs to perform a certain function or service automatically or in response to a request from a user or another device, it may request one or more external electronic devices to perform at least part of the function or service, either instead of or in addition to executing it locally. The one or more external electronic devices that receive the request may perform at least a part of the requested function or service, or additional functions or services related to the request, and transmit the result of execution to the electronic device 101. The electronic device 101 may provide the result as at least a part of the response to the request, either as received or after further processing. To this end, technologies such as cloud computing, distributed computing, mobile edge computing (MEC), or client-server computing may be utilized. The electronic device 101 may provide ultra-low latency services by using distributed computing or mobile edge computing. In another embodiment, the external electronic device 104 may include an Internet of Things (IoT) device. The server 108 may be an intelligent server that uses machine learning and/or neural networks. According to one embodiment, the external electronic device 104 or the server 108 may be included in the second network 199. The electronic device 101 may be applied to intelligent services (e.g., smart homes, smart cities, smart cars, or healthcare) based on 5G communication technology and IoT-related technologies.

The server 108 may be connected to the electronic device 101 and may provide services to the connected electronic device 101. Additionally, the server 108 may carry out a user registration process, store and manage various information related to the registered user, and provide various purchase and payment functions related to the service. The server 108 may also share execution data of service applications running on each of a plurality of electronic devices 101 in real time, so that the services can be shared among users. In terms of hardware, the server 108 may have the same configuration as a conventional web server or service server. However, in terms of software, it may include program modules implemented in various programming languages such as C, C++, Java, Python, Golang, or Kotlin, which provide multiple functions. Furthermore, the server 108 typically refers to a computer system that is connected to an unspecified number of clients and/or other servers through an open computer network such as the Internet, receives task requests from such clients or servers, and provides task results accordingly, along with computer software (i.e., a server program) installed thereon. In addition to the aforementioned server program, the server 108 should be understood in a broad sense to include a series of application programs operating on the server 108 and, in some cases, various databases (DBs) constructed internally or externally (hereinafter referred to as “DB”). Accordingly, the server 108 may classify and store user registration information, and various information and data related to a game, in the DB and manage them, wherein such DB may be implemented either internally or externally to the server 108. In addition, the server 108 may be implemented using server programs available on general-purpose server hardware and operating systems such as Windows, Linux, UNIX, or Macintosh. Exemplary examples include Internet Information Server (IIS) for Windows environments, or CERN, NCSA, APACHE, and TOMCAT for UNIX environments, which may be used to implement web services. The server 108 may also be linked to authentication systems and payment systems for user authentication and service-related payment processing.

The first network 198 and the second network 199 refer to a connection structure that enables the exchange of information between respective nodes such as terminals and servers, or a network that connects the server 108 with electronic devices 101 and 104. The first network 198 and the second network 199 may include, but are not limited to, the Internet, a local area network (LAN), a wireless LAN (WLAN), a wide area network (WAN), a personal area network (PAN), 3G, 4G, LTE, 5G, or Wi-Fi. The first network 198 and the second network 199 may be closed networks such as LANs or WANs, but it is preferable that they are open networks, such as the Internet. The Internet refers to a first and second global open computer network structures 198 and 199 that provide various services existing at protocols such as TCP/IP, TCP, and UDP (User Datagram Protocol) and at upper layers of the protocols, including HTTP (HyperText Transfer Protocol), Telnet, FTP (File Transfer Protocol), DNS (Domain Name System), SMTP (Simple Mail Transfer Protocol), SNMP (Simple Network Management Protocol), NFS (Network File Service), and NIS (Network Information Service).

A database may have a general data structure implemented in the storage space (e.g., a hard disk or memory) of a computer system using a database management program (DBMS). The database may be in a storage format that allows for free retrieval (extraction), deletion, editing, and addition of data. The database may be implemented in accordance with the purpose of one embodiment of the present disclosure using a relational database management system (RDBMS) such as Oracle, Informix, Sybase, or DB2; an object-oriented database management system (OODBMS) such as Gemstone, Orion, or O2; or an XML native database such as Excelon, Tamino, or Sekaiju. To achieve its intended functions, the database may include appropriate fields or elements.

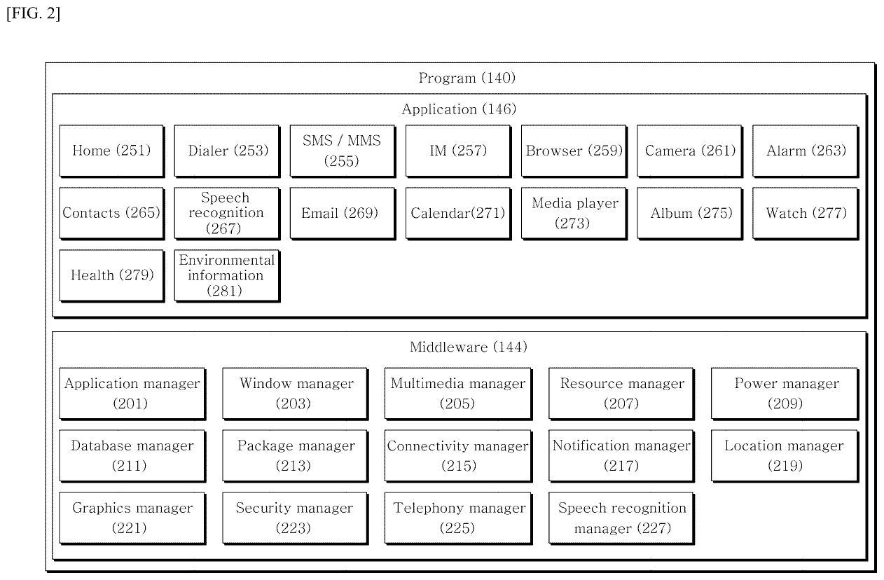

FIG. 2 is a diagram illustrating the configuration of a program 140 according to an embodiment.

FIG. 2 is a block diagram 200 illustrating the program 140 according to various embodiments. According to one embodiment, the program 140 may include an operating system 142, middleware 144, or an application 146 executable on the operating system 142, to control one or more resources of the electronic device 101. The operating system 142 may include, for example, Android™, iOS™, Windows™, Symbian™, Tizen™, or Bada™. At least a part of the program 140 may be preloaded onto the electronic device 101 at the time of manufacture or may be downloaded from or updated via an external electronic device (e.g., an electronic device 102 or 104) or a server 108 during use by a user. All or part of the program 140 may include a neural network.

The operating system 142 may control the management (e.g., allocation or deallocation) of one or more system resources (e.g., processes, memory, or power) of the electronic device 101. In addition or alternatively, the operating system 142 may include one or more driver programs for operating other hardware devices of the electronic device 101, such as an input module 150, an audio output module 155, a display module 160, an audio module 170, a sensor module 176, an interface 177, a haptic module 179, a camera module 180, a power management module 188, a battery 189, a communication module 190, a subscriber identification module 196, or an antenna module 197.

The middleware 144 may provide various functions to the application 146 such that functions or information provided by one or more resources of the electronic device 101 may be used by the application 146. The middleware 144 may include, for example, an application manager 201, a window manager 203, a multimedia manager 205, a resource manager 207, a power manager 209, a database manager 211, a package manager 213, a connectivity manager 215, a notification manager 217, a location manager 219, a graphics manager 221, a security manager 223, a telephony manager 225, or a speech recognition manager 227.

The application manager 201 may manage, for example, the lifecycle of the application 146. The window manager 203 may manage, for example, one or more graphical user interface (GUI) resources used on the screen. The multimedia manager 205 may, for example, identify one or more formats required for playing media files and may perform encoding or decoding of a corresponding media file among the media files using a codec suitable for the selected format. The resource manager 207 may, for example, manage the source code of the application 146 or the memory space of the memory 130. The power manager 209 may, for example, manage the capacity, temperature, or power of the battery 189 and determine or provide information necessary for the operation of the electronic device 101 based on such information. According to one embodiment, the power manager 209 may be linked with a Basic Input/Output System (BIOS) (not shown) of the electronic device 101.

The database manager 211 may, for example, create, retrieve, or modify a database to be used by the application 146. The package manager 213 may, for example, manage the installation or updates of applications distributed in the form of package files. The connectivity manager 215 may, for example, manage wireless or direct connections between the electronic device 101 and an external electronic device. The notification manager 217 may, for example, provide a function to notify the user of the occurrence of a specified event (e.g., an incoming call, message, or alarm). The location manager 219 may, for example, manage location information of the electronic device 101. The graphics manager 221 may, for example, manage one or more graphical effects to be presented to the user or a user interface related thereto.

The notification manager 217 may, for example, provide a function to notify a user of the occurrence of a specified event (e.g., an incoming call, message, or alarm). The location manager 219 may, for example, manage location information of the electronic device 101. The graphics manager 221 may, for example, manage one or more graphical effects to be presented to the user or a user interface related thereto.

The security manager 223 may, for example, provide system security or user authentication. The telephony manager 225 may, for example, manage a voice call function or video call function provided by the electronic device 101. The speech recognition manager 227 may, for example, transmit the user's voice data to the server 108 and receive, from the server 108, a command corresponding to a function to be performed on the electronic device 101 based on at least a part of the voice data, or text data converted based on at least a part of the voice data. According to one embodiment, the middleware 144 may dynamically delete some of the existing components or add new components. According to one embodiment, at least a portion of the middleware 144 may be included as part of the operating system 142 or implemented as separate software independent from the operating system 142.

The application 146 may, for example, include applications such as Home 251, Dialer 253, SMS/MMS 255, IM (Instant Message) 257, Browser 259, Camera 261, Alarm 263, Contacts 265, Speech Recognition 267, Email 269, Calendar 271, Media Player 273, Album 275, Watch 277, Health 279 (e.g., for measuring biometric information such as activity or blood glucose), or Environmental Information 281 (e.g., for measuring barometric pressure, humidity, or temperature). According to one embodiment, the application 146 may further include an information exchange application (not shown) that supports information exchange between the electronic device 101 and an external electronic device. The information exchange application may, for example, include a notification relay application configured to deliver designated information (e.g., calls, messages, or alarms) to an external electronic device, or a device management application configured to manage an external electronic device. The notification relay application may, for example, transmit notification information corresponding to a specified event (e.g., receipt of an email) that occurs in another application (e.g., the email application 269) of the electronic device 101 to an external electronic device. Additionally or alternatively, the notification relay application may receive notification information from the external electronic device and provide it to the user of the electronic device 101.

The device management application may, for example, control the power (e.g., turn-on or turn-off) or functions (e.g., brightness, resolution, or focus) of an external electronic device that communicates with the electronic device 101 or of one or more components thereof (e.g., a display module or camera module of an external electronic device). Additionally or alternatively, the device management application may support installation, deletion, or updating of applications operating on the external electronic device.

Throughout the present specification, the terms “neural network,” “neural network system,” and “network function” may be used interchangeably. A neural network may be composed of a set of interconnected computational units, which are generally referred to as “nodes.” These nodes may also be referred to as “neurons.” A neural network is configured to include at least two or more nodes. The nodes (or neurons) forming the neural networks may be interconnected by one or more “links.”

Within a neural network, two or more nodes connected via links may form a relative relationship of input nodes and output nodes. The concepts of input node and output node are relative; that is, a node that functions as an output node in relation to a certain node may function as an input node in relation to another node, and vice versa. As described above, the input-output relationship between nodes may be formed based on the links. One input node may be connected to one or more output nodes through links, and vice versa.

In an input-output node relationship connected through a single link, the value of the output node may be determined based on the data input into the input node. Here, the link connecting the input node and the output node may have a weight. The weight may be variable and may be adjusted by a user or an algorithm so that the neural network performs a desired function. The edge or link that connects the input and output nodes may have a weight that can be variably applied by the user or algorithm for the neural network to perform the intended function. For example, when one output node is connected to a plurality of input nodes via respective links, the output node may determine its value based on the values input into the input nodes connected thereto and the weights assigned to the respective links corresponding to the input nodes.

As described above, a neural network is formed by interconnecting two or more nodes through one or more links, thereby establishing input-output node relationships within the network. The characteristics of the neural network may be determined based on the number of nodes and links, the interrelationships between the nodes and links, and the values of the weights assigned to the respective links. For example, even if two neural networks have the same number of nodes and links, they may be recognized as different networks if the weights among the links differ.

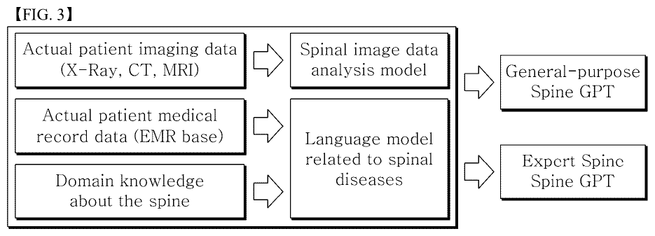

FIG. 3 illustrates a system for assisting in the diagnosis of a patient with spinal disease using artificial intelligence according to an embodiment. The embodiment of FIG. 3 may be combined with various embodiments of the present disclosure.

Referring to FIG. 3, the system for assisting in the diagnosis of a patient with spinal disease using artificial intelligence utilizes actual image data of the patient with spinal disease (e.g., X-ray, CT, MRI), medical record data of the patient with spinal disease (e.g., electronic medical record (EMR) data), and expert knowledge related to the spine (e.g., spine-related knowledge classified into six categories). The image data of the patient with spinal disease may be used to obtain condition information about the patient through a model that analyzes spinal image data, and a natural language processing-based analysis may be performed using a language model trained on the patient's medical data and expert knowledge related to spinal diseases.

That is, the system for assisting in the diagnosis of a patient with spinal disease using artificial intelligence may analyze actual image data and electronic medical record data of the patient to evaluate the spinal condition, and provide natural language processing-based diagnostic support information by utilizing spinal knowledge data. Through this, the system may aim to assist medical professionals by providing both general diagnostic information and specialized diagnostic information about the spine, depending on the target.

In various embodiments of the present disclosure, the system for assisting in the diagnosis of a patient with spinal disease using artificial intelligence may include a system that builds big data by optimizing medical record data of spinal diseases patients (e.g., EMR (electronic medical record) data) and learns diagnostic algorithms developed by experts, thereby improving diagnostic accuracy for spinal patients and enabling appropriate treatment for spinal diseases patients.

In various embodiments of the present disclosure, the system for assisting in the diagnosis of a patient with spinal disease using artificial intelligence may utilize artificial intelligence technology that operates through a mechanism similar to a physician's reasoning process by leveraging the chief complaint of the patient with spinal disease. The chief complaint of a patient with spinal disease refers to the primary issues or symptoms that the patient experiences and expresses. It represents the main reason the patient seeks medical attention and plays an important role in evaluating and diagnosing the patient with spinal disease's health condition.

Here, since the chief complaints of spinal diseases patients used to train artificial intelligence are primarily entered in sentence form, there is a problem in that data collection requires a significant amount of time. In addition, because the method of inputting chief complaints may vary by hospital, or different terms may be used to refer to the same symptoms, there may be difficulties in processing the data. To address these issues, a spinal disease-related natural language processing model trained on chief complaints of a plurality of patients with spinal disease is needed.

In various embodiments of the present disclosure, the system for assisting in the diagnosis of a patient with spinal disease using artificial intelligence may use a natural language processing model trained based on the chief complaints of patients with spinal disease.

Hereinafter, a method in which a server provides diagnostic information about spinal diseases to a user terminal using the natural language processing model related to spinal diseases will be described.

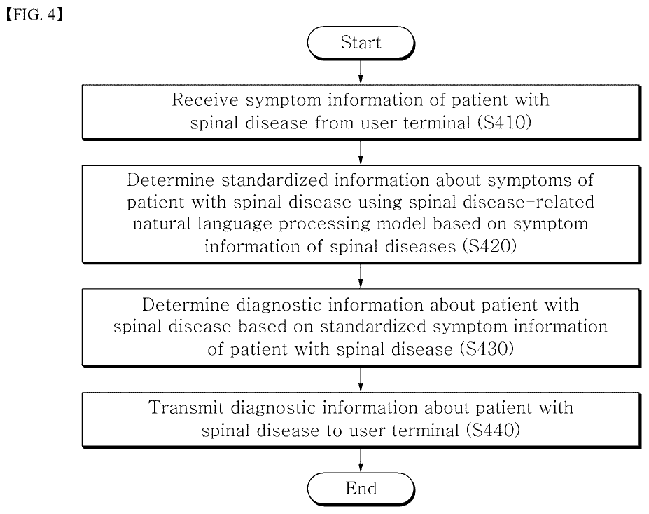

FIG. 4 illustrates a method in which a server provides diagnostic information about spinal diseases to a user terminal according to an embodiment. The embodiment of FIG. 4 may be combined with various embodiments of the present disclosure.

Referring to FIG. 4, in S410, the server may receive symptom information of a patient with spinal disease from a user terminal.

The server may provide diagnostic information about spinal diseases to the user terminal by using the spinal disease-related natural language processing model based on the symptom information of a patient with spinal disease received from the user terminal. For example, the symptom information of the patient with spinal disease may be text data describing various symptoms related to spinal diseases. The symptom information received from the user terminal may, for example, be input in the form of sentences written in natural language, including general vocabulary and/or medical terminology. For example, the server may include the server 108 of FIG. 1.

The user terminal may be a terminal that transmits the spinal disease-related patient information to the server so as to obtain diagnostic information corresponding to the patient's symptom information. For example, the user terminal may transmit the symptom information of the patient with spinal disease to the server by allowing the user to input symptoms related to spinal diseases via a user interface. The user terminal may, for example, access a website operated by the server to provide diagnostic information about spinal diseases using the spinal disease- related natural language processing model. For example, the user terminal may transmit symptom information of the patient with spinal disease to the server by inputting spinal disease-related symptoms into a text or voice input interface window on the web page. When voice is input, the voice data may be converted into text data through various speech-to-text (STT) methods. For example, the user terminal may include the electronic device 101 of FIG. 1.

According to one embodiment, the user terminal may be pre-installed in a plurality of hospitals or medical institutions. In this case, the user terminal may collect various types of information (e.g., a patient's EMR data) in a hierarchical structure. The hierarchical structure may be a method of classifying and storing data in a layered manner based on specific criteria. For example, the user terminal may collect information in a structure where the top level represents the hospital or medical institution, the middle level represents departments or medical teams within each institution, and the bottom level represents the patient's medical records. For instance, the user terminal may set a patient's personal and demographic information as the top level, the patient's health status and treatment history as the middle level, and specific data regarding each health condition and medical record as the bottom level.

According to one embodiment, the user terminal may process the information collected in a hierarchical structure using a federated learning method. That is, the user terminal may process the hierarchically collected information using a local federated learning approach, rather than transmitting the information to the server for processing. Federated learning may be a method in which each terminal independently trains a model and updates data locally in a situation where data is distributed. This allows for the protection of data privacy and the effective utilization of distributed data. For example, each of a plurality of user terminals may train a model based on the data it has collected and may share the parameters of the trained model with other user terminals via a secure communication method. The overall model may be updated based on the shared parameters, and the performance of the model may gradually improve through repeated iterations of this process.

For example, the symptom information of spinal diseases may include a natural language sentence input by the user terminal.

For example, the server may generate at least one sentence vector from at least one natural language sentence included in the symptom information of a patient with spinal disease using a word piece tokenizer. The sentence vector may be a vector in which the text of the natural language sentence is segmented into processable units (e.g., tokens). In other words, each sentence included in the symptom information of the patient with spinal disease may be generated as a sentence vector. For example, at least one sentence vector may include a start token indicating the beginning of the first sentence and a separator token for distinguishing between sentences.

In S420, the server may determine standardized information about the symptoms of a patient with spinal disease based on the symptom information of spinal diseases through the spinal disease-related natural language processing model.

The spinal disease-related natural language processing model may be a model trained to standardize symptom information of patients with spinal diseases based on the chief complaints of a plurality of such patients. That is, since the chief complaints collected from multiple hospitals may use different terms or abbreviations to refer to body parts, types of symptoms, and levels of symptom severity depending on the hospital or the diagnosing physician, the spinal disease-related natural language processing model may be trained to convert the names of body parts, symptom types, and symptom severity included in the chief complaints of spinal diseases patients into standardized values.

The standardized information about the symptoms of a patient with spinal disease may include identifier values matched to standardized terms for body parts, types of symptoms, and levels of symptom severity. For example, a plurality of standardized terms and corresponding identifier values for each of the standardized terms may be preset in the server. For example, the standardized information about the patient's symptoms may include standardized information for the affected body part, the type of symptom, and the severity of the symptom. The standardized information for the affected body part may include an identifier value matched to a standardized term for each of a plurality of body parts. The standardized information for the symptom type may include an identifier value matched to a standardized term for the type of symptom. The standardized information for the symptom severity may include an identifier value matched to a standardized term for the level of symptom severity.

For example, if the standardized term is “cervical spine,” and the symptom information of the patient with spinal disease includes general terms referring to the cervical spine, such as “neck,” “neck bone,” or “neck vertebra,” or professional terms such as “C-spine,” “C1,” “C2,” or “cervical spine,” the standardized information for the affected body part may include the identifier value corresponding to “cervical spine.”

For example, if the standardized term is “acute pain,” and the symptom information of the patient with spinal disease includes general expressions such as “sudden pain,” “unexpected pain,” or “temporary pain,” or professional terms such as “acute pain” or “AP,” the standardized information for the type of symptom may include the identifier value corresponding to “acute pain.”

For example, if the standardized term is “mild pain,” and the symptom information of the patient with spinal disease includes general expressions such as “slight pain,” “minor discomfort,” or “somewhat aching,” or professional terms such as “mild pain” or “MP,” the standardized information for the type of symptom may include the identifier value corresponding to “mild pain.” In this case, the identifier value matched to the standardized term for symptom severity may be a value representing the severity of symptoms on a scale from 0 to 10, where a value closer to 10 may indicate a higher level of severity.

For example, the spinal disease-related natural language processing model may use a Bidirectional Encoder Representations from Transformers (BERT) model. BERT may be a pre-training model for natural language processing with bidirectional characteristics based on a transformer architecture. That is, BERT may compute embeddings for each word by considering the context from both directions of an input sentence. BERT includes a pre-training phase and a fine-tuning phase; during the pre-training phase, the language model may be trained using a large-scale text dataset. In the fine-tuning phase, the pre-trained model may be further tuned using data specific to a particular natural language processing task. During the pre-training phase, BERT may mask words representing body parts, symptom types, and symptom severity in the input sentence and perform operations to restore them. This may improve BERT's ability to understand context and convert masked words into standardized terms. Additionally, BERT may perform a next sentence prediction (NSP) operation to determine whether two sentences are connected. This allows BERT to learn inter-sentence relationships and enhance its performance on natural language processing tasks.

For example, the server may, for example, generate at least one sentence vector from at least one natural language sentence included in the symptom information of a patient with spinal disease, and determine standardized information about the patient's symptoms by inputting the at least one sentence vector into the BERT-based spinal disease-related natural language processing model.

According to one embodiment of the present disclosure, the BERT-based spinal disease-related natural language processing model may be trained based on the chief complaints of a plurality of patients with spinal disease.

For example, the BERT model may include a token embedding layer, segment embedding layer, and position embedding layer for converting the chief complaints of a plurality of patients with spinal disease into a plurality of embedding vectors.

For example, for each chief complaint of a plurality of patients with spinal disease, a natural language sentence constituting the chief complaint may be converted into a sentence vector including a plurality of tokens through tokenization. The chief complaint may include a natural language sentence in which a patient with spinal disease expresses symptoms using general vocabulary and/or a natural language sentence in which a physician expresses the patient's symptoms using medical terminology. For example, the chief complaints of a plurality of patients with spinal disease may be pre-collected information for training the spinal disease-related natural language processing model. For example, spelling errors and unnecessary characters may be removed from each of the natural language sentences that constitute the chief complaints of a plurality of patients with spinal disease.

For example, in each of the plural sentence vectors, tokens corresponding to body parts, types of symptoms, and levels of symptom severity may be annotated with standardized terms.

Additionally, for example, mapping tables for body parts, types of symptoms, and levels of symptom severity may be pre-stored in the server. The mapping table may be a table in which words having the same meaning are mapped to each of a plurality of standardized terms. For example, a single standardized term may be mapped to multiple general expressions and multiple professional terms. The server may determine, based on the mapping table, the tokens corresponding to body parts, symptom types, and symptom severity in each of the plurality of sentence vectors. For example, the server may annotate each token with a standardized term mapped thereto based on the mapping table.

For example, the server may determine, based on the mapping table, tokens corresponding to body parts, types of symptoms, and levels of symptom severity in each of the plural sentence vectors. For example, the server may annotate the tokens corresponding to body parts, symptom types, and symptom severity with the standardized terms mapped to those tokens based on the mapping table.

For example, all sentence vectors included in a single chief complaint may be combined to generate a complaint vector for each of the plural patients with spinal disease.

For example, each of the plurality of complaint vectors may undergo masking, through a token embedding layer, on the tokens that have been annotated in each sentence vector. The token embedding layer may, for example, perform masking on the annotated tokens in each sentence vector. The server may, for example, mask the tokens corresponding to body parts, types of symptoms, and levels of symptom severity through the token embedding layer. That is, the server may replace the tokens corresponding to body parts, symptom types, and symptom severity with mask tokens through the token embedding layer. In this case, the server may replace the token for a body part with a first mask token, the token for a symptom type with a second mask token, and the token for symptom severity with a third mask token.

For example, each of the plural complaint vectors may be converted into a plurality of intermediate representation vectors by adding a start token indicating the beginning of the first sentence and a separator token for distinguishing sentences through the token embedding layer. For example, the server may generate an intermediate representation vector by adding a start token and a separator token to the masked complaint vector through the token embedding layer.

For example, the server may generate the plural embedding vectors from the plural intermediate representation vectors by assigning, through the segment embedding layer and the position embedding layer, a segment identifier value for each sentence vector included in the intermediate representation vector and an identifier value indicating the order of each token included in the sentence vector.

For example, the BERT model may be trained through a process of predicting standardized terms for the masked tokens based on the plural embedding vectors.

For example, when the embedding vectors are input into the encoder layer included in the BERT model, the encoder layer may learn by predicting the standardized terms corresponding to the mask tokens.

In S430, the server may determine diagnostic information for a patient with spinal disease based on the standardized information about the patient's symptoms.

The diagnostic information for a patient with spinal disease refers to information diagnosed based on the patient's symptoms and may, for example, include a diagnosis name and a management method corresponding to the diagnosis name.

According to one embodiment, the server may determine items of required information that correspond to the standardized information about the symptoms of a patient with spinal disease. The required information may be additional information needed to determine a diagnosis name based on the standardized information of the patient with spinal disease. The items of required information may refer to categories of information additionally needed to determine a diagnosis name from the standardized information of the patient with spinal disease. For example, the items of required information may include items related to personal information, medical history, and social history. For instance, the personal information items may include the patient's gender and age. The medical history items may include past surgical history, previous diagnosis names, previous treatment methods, presence or absence of gait disturbance, type of gait, list of diseases, currently taken medications, and results of physical tests. The social history items may include smoking status and amount, frequency and amount of alcohol consumption, exercise frequency, and lifestyle habits.

For example, for each piece of standardized information corresponding to a plurality of patients with spinal disease, items of required information matching the standardized information may be preset. Based on the standardized information of a patient with spinal disease, a plurality of diagnosis names having a diagnosis probability equal to or greater than a preset threshold may be determined. For instance, the preset probability may be 70 percent. That is, a plurality of diagnosis names with high diagnosis probability for the standardized information of a patient with spinal disease may be collected from external terminals, along with the items of additional required information needed to distinguish among the diagnosis names. Through this, the server may match the additional required information items to the standardized information of the patient with spinal disease so as to distinguish among the plural diagnosis names. For example, the items of required information may be updated at a preset interval.

For example, the server may request required information from the user terminal based on the items of required information. The server may, for example, receive the required information from the user terminal.

For example, by performing data preprocessing on the standardized information about the symptoms of a patient with spinal disease, a symptom vector that includes a value for at least one body part, a value for the type of symptom corresponding to each body part, and a value for the severity of the symptom corresponding to each symptom type may be generated.

The value for at least one body part may include an identifier value matched to a standardized term for the corresponding body part. The value for the type of symptom corresponding to each body part may include an identifier value matched to a standardized term for the symptom type. The value for the severity of the symptom corresponding to each symptom type may include an identifier value matched to a standardized term for the symptom severity.

For example, by performing data preprocessing on the required information for a patient with spinal disease, a patient vector that includes a value for gender, a value for age, a value related to gait, a value related to medical history, a value related to medications taken, and a value related to physical tests may be generated.

The value related to gait may include a value indicating the presence or absence of a gait disturbance and a value indicating the type of gait. The value indicating the presence of a gait disturbance may be set to 1 if a gait disturbance is present, and to 0 if there is no gait disturbance. The gait type may represent the walking pattern of a patient with spinal disease. For example, the value for gait type may include a value representing one gait type selected from among a plurality of gait types. The plurality of gait types may include, for example, normal gait, limping gait, Trendelenburg gait, spastic gait, steppage gait, ataxic gait, and antalgic gait. In various embodiments of the present disclosure, the gait types may include additional types beyond those listed above. For example, values corresponding to each gait type may be preset in the server.

The value related to medical history may include a value for past surgeries, a value for previous diagnosis names, and a value for previous treatment methods. The value for past surgeries may include an identifier value corresponding to a surgery previously received by the patient with spinal disease. The value for previous diagnosis names may include an identifier value corresponding to a disease previously diagnosed in the patient with spinal disease. The value for previous treatment methods may include an identifier value corresponding to a method by which the patient with spinal disease was previously treated. For example, the treatment methods may include methods related to physical therapy, pharmacological therapy, injection therapy, and non-surgical therapy. A plurality of identifier values for surgeries, diagnosis names, and treatment methods may be preset in the server.

The value related to medications taken may include an identifier value corresponding to a medication being taken by the patient with spinal disease. For example, identifier values for a plurality of medications may be preset in the server.

The value related to physical tests may include a result value for at least one physical test used for diagnosing and evaluating spinal diseases. For example, the physical tests may include a straight leg raise test (SLRT), femoral nerve stretch test, sensory test, Babinski reflex test, and sacroiliac joint (SU) test. The result value for at least one physical test may be set to 1 if the result is positive and to 0 if the result is negative.

For example, among the values for gender, age, gait, medical history, medications taken, and physical tests, any item that is not received from the user terminal may be set to a value of 0.

For example, a diagnosis name for a patient with spinal disease may be determined based on the symptom vector and the patient vector being input into a diagnosis name determination model utilizing a neural network.

For example, among the plurality of management methods assigned to the diagnosis name for a patient with spinal disease, the management method that matches the patient vector may be determined as the management method for the patient with spinal disease.

Additionally, for example, for each diagnosis name, a plurality of patient groups may be generated through clustering based on a plurality of patient vectors, and a management method may be assigned to each patient group. The plural patient groups may be determined using a clustering technique based on the plural patient vectors. Clustering may refer to unsupervised learning that groups data with similar attributes into a fixed number of clusters. For example, the plural patient vectors for clustering may be generated based on the required information for the plural patients with spinal disease who have been assigned the same diagnosis name. Specifically, the plural patient vectors may be reduced to three or fewer dimensions using various dimensionality reduction techniques. The server may, for example, reduce the dimensions of the plural patient vectors to three or fewer using various dimensionality reduction methods. For instance, the server may reduce the patient vectors to three or fewer dimensions using a principal component analysis (PCA) technique. For example, the server may determine the axis of the data that exhibits the highest variance when the plural patient vectors are projected onto the principal component axes, and may reduce the dimensionality using the determined axis. For example, the server may generate a first axis based on the highest variance among the plural patient vectors, generate a second axis orthogonal to the first axis, and then generate a third axis orthogonal to the second axis. When the server projects the original data onto the three generated vector axes, the server may reduce the dimensionality of the original data to a number of dimensions equal to the number of vector axes. Hereinafter, a vector reduced through various dimensionality reduction techniques may be referred to as a dimensionality-reduced vector.

For example, the plural patient groups may be determined based on the plural dimensionality-reduced vectors using the Density-Based Spatial Clustering of Applications with Noise (DBSCAN) algorithm. For example, DBSCAN assumes that a given point belongs to a cluster if it is located near many other points within that cluster, and it uses a radius and a minimum number of points for such calculation. For example, the radius may refer to a distance threshold centered around a specific data point and may be referred to as a dense area. For example, the minimum number of points may indicate how many neighboring points are needed around a point to designate it as a core point. In addition, each point in the dataset may be classified as a core point, a border point, or an outlier point. For example, the server may check the radius for each point and examine how many neighboring points exist within that range. If at least m points are found within the radius, the server may determine the point as a core point. The server may then classify the points within the radius of the core point as border points. Points that are not within the radius of any core point may be determined as outlier points, which may be excluded from the corresponding cluster. Furthermore, if the distance between two core points is less than the radius, the server may classify them into the same cluster. Through this process, the server may classify the plural patient groups by considering not only age and gender but also medical history and social history.

For example, the management method for a patient with spinal disease may include a first type of management method written for the general public and a second type of management method written for medical professionals.

For example, the diagnostic information for a patient with spinal disease may include a diagnosis name and a management method for the patient with spinal disease.

For example, the diagnosis name determination model may be trained based on plural symptom vectors, plural patient vectors, plural reference vectors, and plural correct diagnosis names.

In S440, the server may transmit the diagnostic information for a patient with spinal disease to the user terminal.

For example, the server may transmit the diagnostic information to the user terminal by displaying the text of the diagnostic information in an interface window on a website.

Additionally, for example, the management method for a patient with spinal disease may be determined as one of the plural types based on the number of professional terms included in the symptom information of the patient with spinal disease.

For example, the server may determine the number of professional terms included in at least one sentence vector based on a mapping table. The at least one sentence vector may be generated from the symptom information of the patient with spinal disease.

For example, the server may set the type of management method for the patient with spinal disease as the first type based on the number of professional terms being less than or equal to a preset number. Conversely, the server may determine the type of management method as the second type based on the number of professional terms exceeding the preset number.

For example, the preset number may be determined by multiplying the number of sentence vectors by a reference value corresponding to the diagnosis name. Here, the number of sentence vectors may be the number of sentences included in the symptom information of the patient with spinal disease. The reference value corresponding to each diagnosis name may be preset in the server for each of the plural diagnosis names. For example, the reference value corresponding to a diagnosis name may be the average number of professional terms included in the chief complaints collected for that diagnosis name. Through this, the server may flexibly set the preset number based on both the diagnosis name and the number of sentences included in the symptom information, rather than using a fixed value. Accordingly, the server may more accurately assess the level of medical knowledge of the user terminal that transmitted the symptom information of the patient with spinal disease.

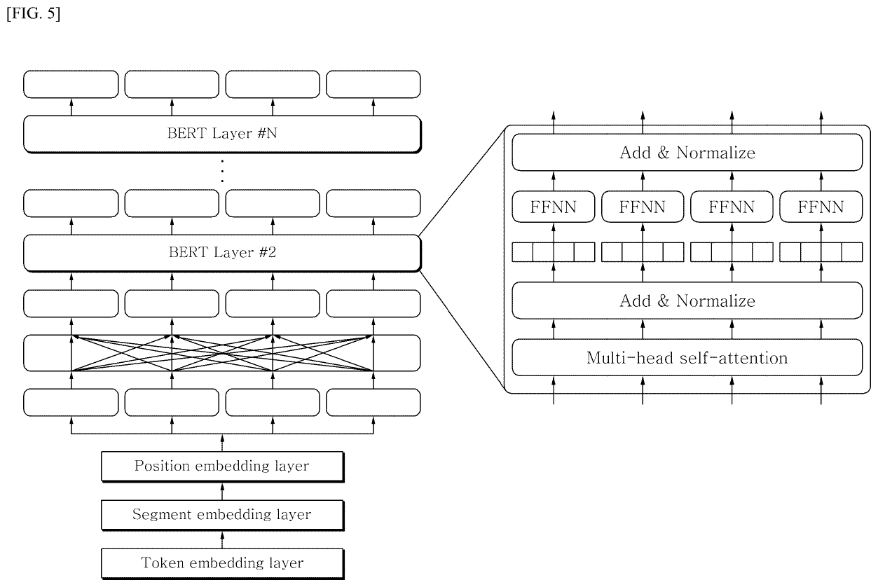

FIG. 5 illustrates a spinal disease-related natural language processing model according to an embodiment. The embodiment of FIG. 5 may be combined with various embodiments of the present disclosure.

Referring to FIG. 5, the spinal disease-related natural language processing model may use a BERT model. For example, the BERT model may include a token embedding layer, a segment embedding layer, a position embedding layer, and a BERT layer. The BERT layer may, for example, include a plurality of encoder layers and output layers.

The token embedding layer may convert tokenizer-preprocessed text into token vectors that include tokens, for a plurality of text inputs. For example, by preprocessing the plural texts through a tokenizer via the token embedding layer, a token vector may be generated by tokenizing a sentence into a plurality of morphemes. In addition, by using a masked language model on the token vector, the encoder layer may learn by inserting the full sentence into the model via a given sequence and predicting the standardized terms corresponding to masked tokens. After training on the sentence, the model may represent the sentence as a sequence of tokens, and the portions to be masked in the token sequence may be set as mask tokens.

For example, the token embedding layer may perform embedding on the sentence vectors using a word piece embedding method, which is a subword tokenizer technique that splits text into units smaller than words, for each of one or two sentence vectors among the plural sentence vectors. For instance, the token embedding layer may tokenize the text of the sentence vectors and convert them into the corresponding indices of the word pieces. A start token may be added at the beginning of the first sentence, and a separator token may be added to distinguish between sentence vectors.

The segment embedding layer may reconstruct the tokenized words into a single sentence. For example, when two sentence vectors are input, the segment embedding layer may assign a value of 0 to the tokens included in the first sentence vector and a value of 1 to the tokens included in the second sentence vector. That is, the segment identifier value may be set to 0 for the first sentence vector and to 1 for the second sentence vector. For example, the segment embedding layer may assign a value of 0 to tokens belonging to odd-numbered sentence vectors and a value of 1 to tokens belonging to even-numbered sentence vectors.

The position embedding layer may add a value indicating the position of each morpheme to a token vector that has been tokenized from a single sentence into a plurality of morphemes. In other words, an identifier value representing the order of each token may be assigned. For example, the tokens may be converted into Q token vectors representing queries, K token vectors representing keys, and V token vectors representing values.

For example, when two sentence vectors are input, the segment embedding layer may assign a value of 0 to the tokens included in the first sentence vector and a value of 1 to the tokens included in the second sentence vector. That is, the segment embedding layer may assign a value of 0 to tokens belonging to odd-numbered sentence vectors and a value of 1 to tokens belonging to even-numbered sentence vectors. The position embedding layer may represent the position information of all tokens in the order of their positions within the sentence. If the maximum sentence length is set to 128, 128 position embedding vectors may be trained. For instance, if the sentence length is 4, four position embedding vectors may be trained as 0, 1, 2, and 3 for each corresponding token.

For example, the plural embedding vectors may be input into the BERT layer.

The BERT layer may, for example, fine-tune the BERT model by integrating the hidden tokens. The plural encoder layers may include multi-head self-attention, a position-wise feed- forward network, and an add-and-normalization layer. For example, the multi-head self-attention mechanism may connect multiple attention layers in parallel, compute query (Q), key (K), and value (V) for each input vector, and measure the similarity among them to determine how closely each input vector is related to the others.

For example, the encoder layer may use scaled dot-product attention to determine the relevance between each word included in the chief complaint. The encoder layer may determine the relationship between a pair of words based on the scaled dot-product attention as represented by Equation 1 below.

$$Attention ( Q , K , V ) = {softmax}_{k} ( \frac{{QK}^{T}}{\sqrt{{d}_{k}}} ) V \qquad [ Equation 1 ]$$

In Equation 1, Q is a vector corresponding to the hidden state from the previous layer of the encoder layer, K is a vector corresponding to the output state of the encoder layer, and V is also a vector corresponding to the output state of the encoder layer. The dk may be the dimensionality of the K vector. In other words, Q may represent the token in the encoder layer that receives the influence, K may represent the token in the encoder layer that exerts the influence, and V may represent the token in the encoder layer to which the weight of that influence is applied.

That is, the server may determine a pairwise matrix for the words in a sentence by computing the dot product between Q and K using cosine similarity, dividing it by the square root of the dimensionality of the K vector, and applying a softmax function. For example, the encoder layer may output the standardized term with the highest probability for a masked token in the input vector using the pairwise matrix of the words in the sentence.

For example, a position-wise feed-forward network may consist of two linear transformations and an activation function, applied independently at each token position. The activation function may be a Rectified Linear Unit (ReLU) function. For instance, the position-wise feed-forward network may perform a linear transformation on the input vector through a first fully connected layer, learn complex patterns through the ReLU function, and then perform another linear transformation on the input vector through a second fully connected layer. For example, the position-wise feed-forward network may be applied separately to each token position to help the model better understand the context.

For example, the add-and-normalization layer may prevent loss of input information through residual connections and stabilize learning through a normalization layer. The residual connection may sum the input of the encoder layer with the output of the multi-head self-attention. The normalization layer may normalize the result of the summation.

For example, the output layer may determine the standardized term with the highest probability as the output value for each token corresponding to a body part, symptom type, and symptom severity included in the input vector by applying a softmax function.

Through this process, the server may train the spinal disease-related natural language processing model to convert various inconsistent terms referring to body parts, symptom types, and symptom severity included in each chief complaint into standardized terms, based on the pre- collected chief complaints of a plurality of patients with spinal disease. Accordingly, the server may obtain standardized information about the symptoms of patients with spinal disease based on the chief complaints of the patients, and may subsequently utilize the standardized information as training data in various artificial intelligence systems.

Additionally, according to one embodiment, the diagnosis name determination model may use a convolutional neural network (CNN)-based neural network. For example, the CNN-based neural network may include an input layer, one or more hidden layers and an output layer.

For example, the diagnosis name determination model may be trained based on a plurality of symptom vectors, a plurality of patient vectors, a plurality of reference vectors, and a plurality of correct diagnosis names.

For example, training data composed of a plurality of symptom vectors, a plurality of patient vectors, a plurality of reference vectors, and a plurality of correct diagnosis names may be input into the input layer, pass through one or more hidden layers and the output layer, and be output as an output vector. The output vector may then be input into a loss function layer connected to the output layer. The loss function layer may output a loss value by using a loss function that compares the output vector with the correct vector for each training data point, and the parameters of the neural network may be trained in a direction that minimizes the loss value.

One or more hidden layers may include one or more convolution layers and one or more pooling layers. For example, in the convolution layer, a plurality of symptom vectors and a plurality of patient vectors may be filtered, and a feature map may be generated through the convolution layer.

For example, based on the feature map generated in the pooling layer, fixed vectors related to features may be selected for dimensionality reduction, and sub-sampling may be performed on the generated feature map to extract features related to the plural reference vectors from the vectorized time-series data. The pooling layer may be, for example, a max pooling layer that extracts the maximum value. The pooling layer may be, for example, an average pooling layer that extracts the mean value. In this case, the parameters of the neural network may include parameters, such as the size of the feature map, the size of the filter, the depth, the stride, and the zero padding, related to the convolution layer and the pooling layer.

For example, one symptom vector and one patient vector may form one training data set along with one correct diagnosis name. In this case, a plurality of training data sets may be pre-stored.

For example, the plural symptom vectors and plural patient vectors may, for example, be generated based on the chief complaints and required information of each of a plurality of patients with spinal disease.

For example, the plural reference vectors may serve as reference vectors for comparison with the symptom vector and patient vector of a patient with spinal disease. For example, the plural reference vectors may include a reference symptom vector and a reference patient vector for each of the plural diagnosis names.

For example, the correct diagnosis name may include a value corresponding to the diagnosis name that was ultimately confirmed for the patient with spinal disease whose chief complaint and required information were collected. The plural correct diagnosis names may, for example, represent actual confirmed diagnosis names for the plural patients with spinal disease. For example, values representing the plural diagnosis names may be preset in the server.

For example, the diagnosis name determination model may construct each of the plural symptom vectors and plural patient vectors as a single input vector, determine an initial similarity for each of the plural reference vectors with respect to each input vector, and identify, for each input vector, the reference vector with the highest initial similarity. For example, the diagnosis name determination model may determine whether the diagnosis name of the most similar reference vector matches the correct diagnosis name assigned to the input vector. If they do not match, the model may adjust the weights used to calculate the initial similarity until the correct diagnosis name is determined.

Additionally, the initial similarity between the input vector and the reference vector input into the diagnosis name determination model may be determined, for example, by Equation 2 below. In this case, the input vector may include a symptom vector and a patient vector, and the reference vector may include a reference symptom vector and a reference patient vector.

$$simS\_ini = {K}_{1} = \frac{{w}_{s} * {\sum }_{i = 1}^{n} {w}_{i} * {S}_{i} * {R}_{1 , i}}{{K}_{2} + {\sum }_{i = 1}^{n} {S}_{i}^{2} * {\sum }_{i = 1}^{n} R \text{?}} + \frac{{w}_{p} * {\sum }_{j = 1}^{m} {P}_{j} * {R}_{2 , j}}{{K}_{3} + {\sum }_{j = 1}^{m} P \text{?} * {\sum }_{j = 1}^{m} {R}_{2 , j}^{3}} \qquad [ Equation 2 ]$$

$$\text{?} \text{indicates text missing or illegible when filed}$$

In Equation 2, simS_ini denotes the initial similarity between the input vector and the reference vector, n denotes the dimensionality of the symptom vector and the reference symptom vector, Sᵢ denotes the value of the i-th dimension of the symptom vector, R_{1,i} denotes the value of the i-th dimension of the reference symptom vector, wᵢ denotes the weight for the i-th dimension of the reference symptom vector, m denotes the dimensionality of the patient vector and the reference patient vector, Pⱼ denotes the value of the j-th dimension of the patient vector, R_{2,j} denotes the value of the j-th dimension of the reference patient vector, wₛ denotes the weight for the symptom vector of the diagnosis name matched to the reference vector, wₚ denotes the weight for the patient vector of the diagnosis name matched to the reference vector, and K1, K2, and K3 may be correction constants.

For example, the weight for each dimension of the reference symptom vector may be a value greater than 0 and less than 1. For example, based on a plurality of chief complaints collected for a single diagnosis name, the frequency of standardized terms for body parts, types of symptoms, and symptom severity may be determined. Then, based on the frequency of the standardized terms for body parts, symptom types, and symptom severity for that diagnosis name, the weight for each dimension of the reference symptom vector may be assigned. For example, the weight for each dimension of the reference symptom vector may be determined by adding a correction value to the ratio of the frequency of a particular standardized term to the total frequency.

For example, the weight for the symptom vector and the weight for the patient vector of the diagnosis name matched to the reference vector may each be a value greater than 0 and less than 1. The sum of the weight for the symptom vector and the weight for the patient vector of the diagnosis name matched to the reference vector may be equal to 1.

For example, K₁, K₂ and K₃ may be correction constants that are pre-stored in the server.

By utilizing data for training the diagnosis name determination model and using the initial similarity between the input vector and the reference vector, the diagnosis name determination model may be trained to determine diagnosis names more accurately.

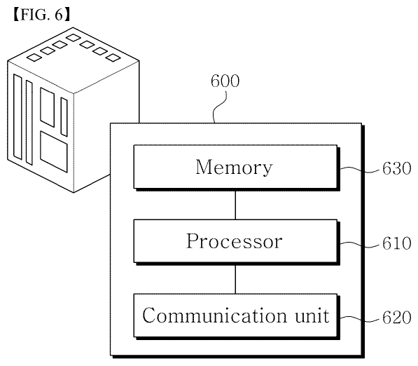

FIG. 6 illustrates a block diagram illustrating the configuration of a server according to one embodiment. The embodiment of FIG. 6 may be combined with various embodiments of the present disclosure.

As illustrated in FIG. 6, a server 600 may include a processor 610, a communication unit 620, and a memory 630. However, not all components shown in FIG. 6 are essential components of the server 600. The server 600 may be implemented with more components than those shown in FIG. 6, or with fewer components than those shown in FIG. 6. For example, in some embodiments, the server 600 may further include a user input interface (not shown), an output unit (not shown), or other components in addition to the processor 610, communication unit 620, and memory 630.

The processor 610 generally controls the overall operations of the server 600. The processor 610 may include one or more processors to control other components included in the server 600. For example, by executing programs stored in the memory 630, the processor 610 may comprehensively control the communication unit 620, the memory 630, and other components. In addition, by executing the programs stored in the memory 630, the processor 610 may perform the functions of the server 600 as described in FIGS. 3 to 5.

The communication unit 620 may include one or more components that enable the server 600 to communicate with other devices (not shown) and servers (not shown). The other device may be a computing device like the server 600 or a sensing device, but is not limited thereto. The communication unit 620 may receive user input from another electronic device via a network or receive data stored in an external device.

For example, the communication unit 620 may transmit and receive messages to establish a connection with at least one device. The communication unit 620 may transmit information generated by the processor 610 to at least one device connected to the server. The communication unit 620 may receive information from at least one device connected to the server. The communication unit 620 may also transmit information related to the received data in response to the received input.

The memory 630 may store programs for processing and controlling the operations of the processor 610. For example, the memory 630 may store information entered into the server or information received from another device via a network. In addition, the memory 630 may store data generated by the processor 610. The memory 630 may also store information input into or output from the server 600.

The memory 630 may include at least one type of storage medium, such as flash memory type, hard disk type, multimedia card micro type, card-type memory (e.g., SD or XD memory), Random Access Memory (RAM), Static Random Access Memory (SRAM), Read-Only Memory (ROM), Electrically Erasable Programmable Read-Only Memory (EEPROM), Programmable Read-Only Memory (PROM), magnetic memory, magnetic disk, or optical disk.

According to embodiments, a server can use a natural language processing model trained to standardize symptom information of a plurality of patients with spinal disease based on chief complaints of the plural patients, thereby enabling general users to easily check their diagnostic information about spinal diseases through natural language sentences. Furthermore, medical professionals can also check diagnostic information about patients through natural language sentences to assist in diagnosis.

According to embodiments, since the server can obtain standardized information from the text of chief complaints in electronic medical records (EMRs) using the spinal disease-related natural language processing model, it can significantly reduce the time and effort required for data collection and preprocessing, and improve the usability and accessibility of the data by converting unstructured data into a structured format.

Effects obtainable from the embodiments are not limited to those described above, and other effects not mentioned may be clearly derived and understood by those skilled in the art based on the detailed description provided above.

The embodiments described above may be implemented as a hardware component, a software component, and/or a combination of hardware components and software components. For example, the apparatus, methods and components described in the embodiments may be achieved using one or more general purpose or special purpose computers, such as, for example, a processor, a controller, an arithmetic logic unit (ALU), a digital signal processor, a microcomputer, a field programmable gate array (FPGA), a programmable logic unit (PLU), a microprocessor, or any other device capable of executing and responding to instructions. The processing device may execute an operating system (OS) and one or more software applications executing on the operating system. In addition, the processing device may access, store, manipulate, process, and generate data in response to execution of the software. For ease of understanding, the processing apparatus may be described as being used singly, but those skilled in the art will recognize that the processing apparatus may include a plurality of processing elements and/or a plurality of types of processing elements. For example, the processing apparatus may include a plurality of processors or one processor and one controller. Other processing configurations, such as a parallel processor, are also possible.

The software may include computer programs, code, instructions, or a combination of one or more of the foregoing, configure the processing apparatus to operate as desired, or command the processing apparatus, either independently or collectively. In order to be interpreted by a processing device or to provide instructions or data to a processing device, the software and/or data may be embodied permanently or temporarily in any type of a machine, a component, a physical device, a virtual device, a computer storage medium or device, or a transmission signal wave. The software may be distributed over a networked computer system and stored or executed in a distributed manner. The software and data may be stored in one or more computer-readable recording media.

The methods according to the embodiments of the present disclosure may be implemented in the form of a program command that can be executed through various computer means and recorded in a computer-readable medium. The computer-readable medium can store program commands, data files, data structures or combinations thereof. The program commands recorded in the medium may be specially designed and configured for the present disclosure or be known to those skilled in the field of computer software. Examples of a computer-readable recording medium include magnetic media such as hard disks, floppy disks and magnetic tapes, optical media such as CD-ROMs and DVDs, magneto-optical media such as floptical disks, or hardware devices such as ROMs, RAMs and flash memories, which are specially configured to store and execute program commands. Examples of the program commands include machine language code created by a compiler and high-level language code executable by a computer using an interpreter and the like. The hardware devices described above may be configured to operate as one or more software modules to perform the operations of the embodiments, and vice versa.

Although the present disclosure has been described with reference to limited drawings, it should be understood by those skilled in the art that various technical changes and modifications may be made therein. For example, the described techniques may be performed in a different order than the described methods, and/or components of the described systems, structures, devices, circuits, etc., may be combined in a manner that is different from the described method, or appropriate results may be achieved even if replaced by other components or equivalents.

Therefore, other embodiments, other examples, and equivalents to the claims are within the scope of the following claims.

What is claimed is:

1. A method of providing diagnostic information about spinal diseases to a user terminal by a server, the method comprising:
receiving symptom information of a patient with spinal disease from the user terminal, wherein the symptom information of the patient with spinal disease comprises a natural language sentence input by the user terminal;
determining standardized information about the symptoms of the patient with spinal disease through a natural language processing model related to spinal diseases, based on the symptom information of the patient with spinal disease, wherein the spinal disease-related natural language processing model is a model trained to standardize symptom information of a patient with spinal disease based on chief complaints of a plurality of patients with spinal disease, wherein the standardized information about the symptoms of the patient with spinal disease comprises standardized information on a body part where the symptom occurs, standardized information on a symptom type, and standardized information on symptom severity;
determining diagnostic information about the patient with spinal disease based on the standardized information about the symptoms of the patient with spinal disease; and
transmitting the diagnostic information about the patient with spinal disease to the user terminal.

2. The method according to claim 1, wherein in the spinal disease-related natural language processing model, a Bidirectional Encoder Representations from Transformers (BERT) model is used,
the BERT model comprises a token embedding layer, segment embedding layer, and position embedding layer for converting chief complaints of a plurality of patients with spinal disease into a plurality of embedding vectors,
for each chief complaint of the plural patients with spinal disease, a natural language sentence constituting the chief complaint is tokenized into a sentence vector comprising a plurality of tokens,
annotation is performed on the tokens corresponding to body part, symptom type, and symptom severity in each sentence vector with a standardized term,
all sentence vectors comprised in one chief complaint are combined to generate a complaint vector for each of the chief complaints of the plural patients with spinal disease,
for each of the complaint vectors, masking is performed on the tokens annotated in each sentence vector through the token embedding layer, and a start token indicating beginning of a first sentence and a separator token for separating sentences are added to generate a plurality of intermediate representation vectors,
each of the plural intermediate representation vectors is converted into a plurality of embedding vectors by assigning a segment identifier value for each sentence vector in the embedding vectors and an order identifier value for each token in the sentence vector through the segment embedding layer and the position embedding layer, and
the BERT model is trained through a process of predicting a standardized term for a masked token based on the plural embedding vectors.

3. The method according to claim 2, further comprising:
determining an item of required information matching the standardized information about the symptoms of the patient with spinal disease;
requesting the required information from the user terminal based on the item of required information; and
receiving the required information from the user terminal,
wherein the required information is additional information required to determine a diagnosis name from the standardized information of the patient with spinal disease,
for each standardized information of a plurality of patients with spinal disease, items of required information matching standardized information are preset,
by performing data preprocessing on standardized information about symptoms of the patient with spinal disease, a symptom vector comprising a value for at least one body part, a value for symptom type by body part, and a value for symptom severity by symptom type is generated,
by performing data preprocessing on the required information of the patient with spinal disease, a patient vector comprising a value for gender, a value for age, a value related to gait, a value related to medical history, a value related to medications taken, and a value related to physical tests is generated,
a diagnosis name for the patient with spinal disease is determined based on inputting the symptom vector and the patient vector into a diagnosis name determination model using a neural network,
among a plurality of management methods set for the diagnosis name for the patient with spinal disease, a management method matching the patient vector is determined as a management method for the patient with spinal disease,
the diagnostic information about the patient with spinal disease comprises the diagnosis name and the management method for the patient with spinal discase, and
the diagnosis name determination model is trained based on a plurality of symptom vectors, a plurality of patient vectors, a plurality of reference vectors, and a plurality of correct diagnosis names.
